(19) United States
(12) Patent Application Publication
(10) Pub. No.: US 2015/0151070 A1
(43) Pub. Date: Jun. 4, 2015

(54) CLOSURE METHODS AND DEVICES FOR HEAD RESTRAINTS AND MASKS

(71) Applicant: Boa Technology Inc., Denver, CO (US)

(72) Inventors: James Capra, Steamboat Springs, CO (US); Mark Dutton, Steamboat Springs, CO (US); Aaron Venturini, Denver, CO (US); Ilya Minkin, Denver, CO (US); Anna Kulzick, Denver, CO (US)

(73) Assignee: Boa Technology Inc., Denver, CO (US)

(21) Appl. No.: 14/561,100

(22) Filed: Dec. 4, 2014

Related U.S. Application Data
(60) Provisional application No. 61/911,777, filed on Dec. 4, 2013.

(51) Int. Cl. A61M 16/06

(52) U.S. Cl. CPC A61M 16/0644; A61M 16/0683

(57) ABSTRACT

A system for securing a mask about a user's face includes a mask that is configured to fit about the user's face, a padded member that is postionable on the back of the user's head, and at least one strap that extends from the padded member toward the mask. The system also includes a closure system that is coupled with the mask and with the strap. The closure system includes a tension member, a guide that routes the tension member about the mask and/or the strap, and a tensioning device that is operable to tension the tension member and thereby pull the strap and padded member toward the mask to secure and/or tighten the mask about the user's face.

### CROSS-REFERENCES TO RELATED APPLICATIONS

This Application claims the benefit of U.S. Provisional Application No. 61/911,777, filed on Dec. 4, 2013, and titled “Closure Methods and Devices for Head Restraints and Masks,” the entire disclosure of which is incorporated by reference herein for all purposes.

### BACKGROUND OF THE INVENTION

The present embodiments relate generally to head restraints and masks and more specifically to closure or tightening systems that may be used to tighten a restraint or mask against a user's head.

Head restraints and/or masks are commonly positioned and secured about a user's head. Conventional head restraints and/or masks often employ Velcro® straps and/or buckles in securing the mask or restraint to the head. The Velcro® straps and/or buckles often require the user to grasp the strap or the restraint/mask body to couple the strap and/or buckle and secure the restrain/mask to the head. In some instances, the user is required to use both hands to secure the mask/restraint to the head. Further, in some instances the strap and/or buckle may be positioned behind the user's head and thus, the user may not be able to view the buckle and/or coupling point of the strap. Accordingly, properly fitting a mask and/or restraint to the head may be difficult and/or challenging for users.

### BRIEF SUMMARY OF THE INVENTION

The present invention generally provides systems for closing and tightening a mask or head restraint about a user's head or face. According to one aspect, a system for securing a mask about a user's face includes a mask that is configured to fit about the user's face, a padded member that is postionable on the back of the user's head, and at least one strap that extends from the padded member toward the mask. The system also includes a closure system that is coupled with the mask and with the strap. The closure system includes a tension member, a guide that routes the tension member about the mask and/or the strap, and a tensioning device that is operable to tension the tension member and thereby pull the strap and padded member toward the mask to secure and/or tighten the mask about the user's face.

In some embodiments, the strap is positioned on a first side of the user's head and the system includes an additional strap or straps that are positioned on an opposite side of the user's head. In some embodiments, the tensioning device may be or include a rotatable knob and/or the tension member may include lace. In some embodiments, the tensioning device is positioned on the mask or on the padded member.

According to another aspect, a method for securing a mask about a user's face includes providing a mask having: a mask member, a padded member, at least one strap that extends from the padded member toward the mask, and a closure system that is coupled with the mask and with the strap. The closure system includes: a tension member, a guide that routes the tension member about the mask and/or the strap, and a tensioning device that is operable to tension the tension member. The method also includes positioning the mask member about the user's face, positioning the padded member on the back of the user's head, and operating the tensioning device to tension the tension member and thereby pull the strap and padded member toward the mask to secure and/or tighten the mask about the user's face.

In some embodiments, the strap is positioned on a first side of the user's head and the mask includes another strap that is positioned on an opposite side of the user's head. In some embodiments, the tensioning device includes a rotatable knob and/or the tension member includes lace. In some embodiments, the tensioning device is positioned on the mask and/or on the padded member.

### BRIEF DESCRIPTION OF THE DRAWINGS

The present invention is described in conjunction with the appended figures:

FIG. 1 illustrates an embodiment of a mask that may be fit about a user's head with a closure device.

FIG. 2 illustrates another embodiment of a mask that is fit about a user's head.

FIGS. 3A-D illustrate various embodiments of straps that may be used to couple a mask with a user's head.

FIGS. 4A-D illustrate embodiments of attachment mechanisms for attaching the mask/head restraints to an individual's head.

FIGS. 5A-C illustrate various positioning of a reel assembly about a mask/head restraint.

FIG. 6 illustrates an embodiment of a restraint member that may be coupled with a conventional mask.

FIG. 7 illustrates an embodiment in which the restraint member of a mask assembly is thermoformable to a rear surface of the user's head.

FIG. 8 illustrates an embodiment of a mask that includes an attachment port that is configured for releasably coupling with a reel assembly.

FIG. 9 illustrates an embodiment of a restraining member that may be used to apply a perpendicular and roughly even pressure to the user's head.

FIG. 10 illustrates an embodiment of a strap that may be used to restrain movement of a user's head.

FIG. 11 illustrates a head restraint that may be fit and tensioned about a user's head.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE INVENTION

The embodiments described herein provide closure or tensioning devices for head restraints/masks and methods for using such closure/tensioning devices. For convenience in describing the embodiments, the devices will be generally refered to as closure devices. It should be realized, however, that closure of the head restraints/masks is not required and that in many embodiments, the devices will be used merely to tension lace and/or tighten the head restraints/masks. Also, the term head restraint/mask as used herein is meant to include any article that is fit about or coupled with a head, or any manner of holding or restraining the head from movement. For example, the term head restraint/mask may include various helmets, strap devices (e.g., for a table or bed), facial or head masks, and the like. For convenience in describing the embodiments herein, the disclosure will generally refer to head masks, although it should be realized that the closure devices may be used for essentially any other head restraints.

The embodiments described herein may find particular usefulness in head restraints for medical applications. For example, the devices described herein may be used for restraining an individual's head on an operating table or stretcher. Another specific use for the devices is in fitting medical masks to the individual's head. The masks may includes: sleep apnea masks, oxygen masks, gas masks, and the like. As described in greater detail below, the closure devices may aid in easily fitting such masks to the user's head and in comfortably holding the masks tightly to the head.

The closure devices described herein provide a variety or unique benefits or advantages when compared with conventional head restraints/masks. For example, the closure devices increase the life of the head restraints straps by reducing wear on the straps and/or providing alternative closure methods. For example, conventional head restraint straps that employ Velcro® and elastic, or similar closure mechanisms, may quickly wear out. As the user continually opens and closes the Velcro® straps, dirt, fibers, debris, and even the user's hair may entangle with the Velcro® components and negatively affect future closure and/or hold of the straps. The closure devices described herein do not suffer from such issues and may remove the need for using Velcro® type straps entirely.

The closure devices described herein also provide a dynamic fit for the head masks. For example, using the closure devices described herein, the entire mask may be closed about the user's head simultaneously rather than requiring the closure of individual straps. Further, as the user and/or mask moves, the closure device is able to adjust and compensate for such movement, thereby maintaining the fit and/or tightness of the mask about the user's head. This allows the mask, helmet, or head restraint to move without compromising the fit of the system. The closure devices also simplify adjustment by allowing the user to control multiple straps with a single user interface (e.g., a reel assembly as described below). This may reduce the number of straps and/or adjustment mechanisms needed for the mask since a single closure device can simultaneously pull on, or otherwise tighten, multiple areas that need adjustment. The closure devices also provide improved fit, which helps ensure that the mask is in full contact with the head/face. The result may be fewer gaps between the mask and the user's head/face. When oxygen or other gas is being delivered to the user via the mask, the result may be less leakage of the gas and/or increased delivery of the gas to the user.

The closure devices also provide micro-adjustability of the mask's fit about the head/face. Conventional systems that employ Velcro® or other straps do not provide such micro-adjustability. Rather, with conventional systems, the user typically grips the strap and performs a one-time gross or macro adjustment of the mask. This process may be performed multiple times as the user attempts to adjust (i.e., increase or decrease) the tightness and/or fit of the mask for comfort and/or other reasons. The result is that the fit of conventional masks may be somewhat, but not fully comfortable. The user may stop adjusting the mask when a tolerable, but not necessarily comfortable, fit is achieved. Further, as the user's situation changes (e.g., due to activity, swelling, and the like), the user may wish to adjust the fit of the mask, which necessitates the user uncoupling and recoupling the Velcro® or other straps as described above.

In contrast, the closure devices described herein, allow the user to easily micro-adjust the fit of the masks without requiring the user to uncouple the masks straps. In some embodiments, the micro-adjustment of the mask may be in millimeter, or smaller/larger, increments. The micro-adjustment may act dynamically throughout the system, or in other words, micro-adjustment of one side of the mask may be equally realized on an opposite side of the mask. Accordingly, the user may perform a one-time macro or gross adjustment of the masks and then make any micro-adjustments with the closure device. The micro-adjustability may allow the user to achieve comfort levels with the mask that are far greater than those achievable with conventional mask systems. Further, the user is not required to uncouple and recouple the masks straps to adjust the fit of the mask as conditions change throughout the day due to activity level, swelling, and the like. In many embodiments, the micro-adjustment of the mask may be performed with one hand.

The closure devices also allow one or more components of the mask to be quickly and easily replaced. For example, the mask's components may generally fall into one of two categories: soft good and hard goods. Hard goods typically includes any rigid or firm material, or any components attached thereto, such as the body of the mask, valves, oxygen or gas ports/inlets, cushioning pads, and the like. Soft goods typically includes flexible or soft materials, or any components attached thereto, such as fabrics that wrap around the head, straps, elastic body portions, buckles, and the like. The closure devices described herein may allow one or more of these components (i.e., hard or soft goods) to be easily and quickly replaced. For example, in some embodiments, the closure device may be coupled or attached with the soft good component (e.g., entire head adjustment fit system) to allow the soft good component to be detached from the hard good component (e.g., entire mask system) so that the soft good component can be replaced without requiring replacement of the hard good component. Similarly, in other embodiments, the closure device may be coupled or attached with the hard good component (e.g., mask) to allow the hard good component to be replaced without requiring replacement of the soft good component. Such embodiments may greatly reduce the cost in replacing or altering the mask.

In some embodiments, the closure device may employ unique attachment mechanisms, such as the male and female coupling components described in U.S. patent application Ser. No. 14/071,435, filed Nov. 4, 2013, and titled “Coupling Members for Closure Devices and Methods,” the entire disclosure of which is incorporated by reference herein. The use of such attachment mechanisms may simplify gross or macro adjustment of the mask by allowing the user to easily use one hand to attach and detach the mask about the head/face. In reel assembly based closure devices, the attachment mechanisms may also allow the user to use or take up some of the reel assembly's excess lace when donning and doffing the mask. As such, the user may easily don and doff the mask without excessively operating the reel assembly (i.e., excessively winding the reel assembly's knob).

In addition to, or as an alternative to, the use of the unique attachment mechanisms, the closure devices may use releasable guides that allow a reel assembly's lace to be easily coupled and uncoupled from the guides. As used herein, the term “guides” generally refers to any member that is capable of directing and guiding lace along a lace path. Such guides may be made of either relatively hard and rigid material, such as plastic, metal, and the like, or may be made of relatively soft materials, such as webbing, fabric, and the like. In some embodiments, the guide may be made of both types of material. Such guides are typically made of a low friction material so as to avoid abrading or damaging the lace.

The releasable guides may have an open back or rear surface around which the lace is positioned. The open rear surface may form a channel within which the lace is positioned when coupled with the guide. The open configuration allows the lace to be easily and quickly uncoupled from the guide by removing the lace from the channel. The releasable guides may be used as coupling or attachment points for the mask to allow the mask to be easily donned and doffed. In some embodiments, the releasable guides may be mounted on the hard good (e.g., mask) and lace that is coupled with the soft good may be positioned around the guides to couple the hard and soft good components. In other embodiments, the configuration of the releasable guides and lace may be reversed. These configurations allow the lace to be detached from the guide and mask, such as for periodic replacement (e.g., every 3 months) and the like.

As described in greater detail below, the positioning of the closure device about the mask may be adjusted based on the desired closure or fit, or based on access. For example, in one embodiment the closure device may be positioned on or near the front of the mask. In this position, the closure device may be easily accessed by the user and/or may be more comfortable for the user when sleeping. In another embodiment, the closure device may be positioned on or near the rear surface of the system opposite the mask. In this position, the closure device may be easily accessible to a physician or technician who may be fitting the product. This positioning of the closure device may be preferred in head restraint embodiments. The bilateral location of the closure device (i.e., positioning on the front of the mask or the rear surface opposite the mask) allows the laces to wrap around both sides of the head and equalize tension on each side of the head.

The masks/head restraints may have various installation options in addition to the other aspects described herein. For example, the masks/head restraints may include molded-in detachable female receivers that are configured for coupling with a corresponding male component, such as those described in the '435 application incorporated herein. The masks/head restraints may also include a coupling port or “bayonet” that is configured for releasable attachment with a housing of a reel assembly. In some embodiments, the bayonet may be formed into a body of the mask itself In some embodiments, the soft good components may include welded guides and/or tubing that function as guides for the lace. The welded guides/tubing may allow the lace to be easily coupled with the soft good components. Additional methods of forming lace guides are described in U.S. patent application Ser. No. 14/479,173, filed Sep. 5, 2014, and titled “Guides and Components for Closure Systems and Methods Therefore”, the entire disclosure of which is incorporated by reference herein.

Having described the closure devices and head restraints/masks generally, additional aspects and features of the devices will be more evident in the description of the several embodiments provided herein below.

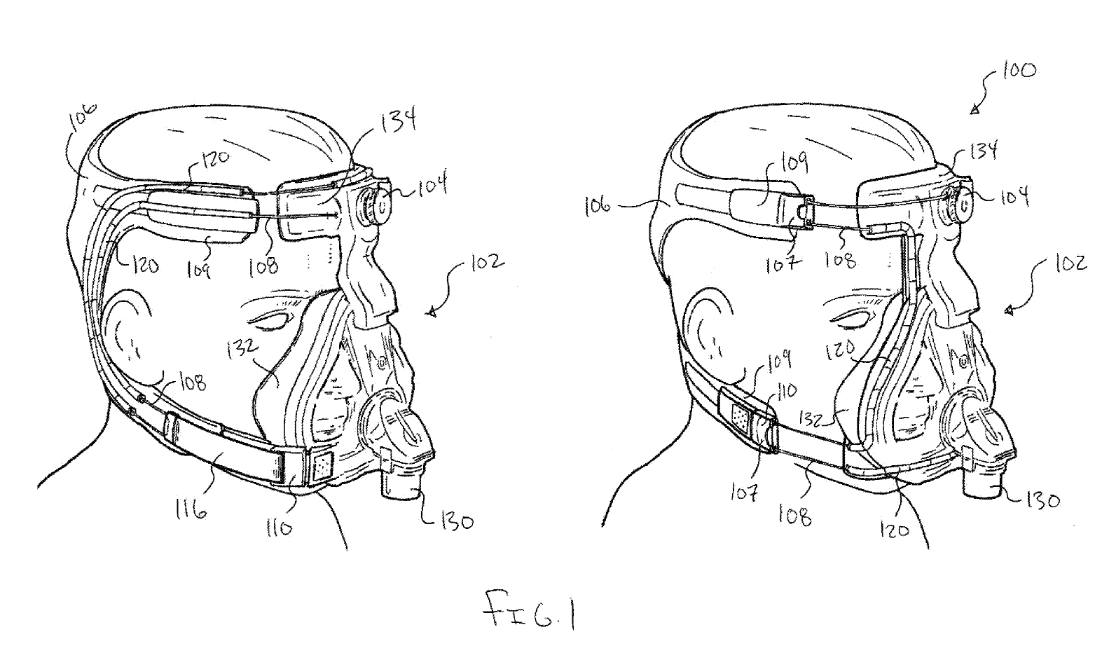

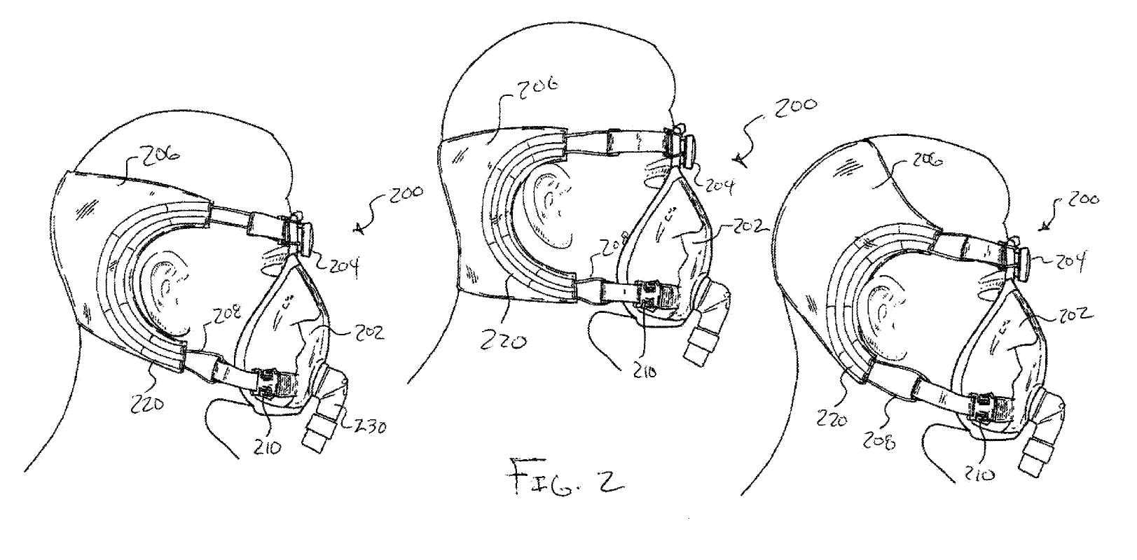

Referring now to FIG. 1, illustrated is an embodiment of a mask 100 that may be fit about a user's head with a closure device. Mask 100 includes a body portion 102 that is fit about the user's nose and mouth so as to deliver oxygen or other gas to the user. Body portion 102 includes an inlet port 130 that connects with tubing (not shown) of the oxygen or gas delivery source. Body 102 also includes a face cushion or pad 132 that presses against the user's face. In many embodiments, pad 132 provides a relatively airtight fit against the user's face to prevent leakage of the oxygen or gas from body portion 102. The airtight fit of the pad 132 may be provided by a restraint member 106 of mask 100, which is typically a large fabric or flexible body that fits or wraps around the rear surface of the user's head. Improper fitting of the mask 100 may result in gaps between the pad 132 and the user's face. These gaps may allow oxygen or gas to escape or leak from mask 100.

Body portion 102 also includes a head cushion 134 that presses against the user's forehead. The restraint member 106 includes a main body portion and a plurality of arm portions 109 that extend towards the body portion 102 and/or head cushion 134. The arm portions 109 and restraint member 106 may be coupled with the head cushion 134 and/or body portion 102 via one or more buckles 110. The restraint member 106 is configured to pull the mask 100 tightly against the user's face to prevent gaps from developing between the user's face and the pads 132. To pull the mask 100 tightly against the user's face, mask 100 includes a reel assembly 104 that is operationally coupled with lace 108 that is in turn coupled with restraint member 106 and arm portions 109. As shown in FIG. 1, restraint member 106 and/or arm portions 109 may include tubing 120 through which the lace 108 is inserted. The tubing 120 may guide the lace 108 along restraint member 106 and/or arm portions 109 between body portion 102 and head cushion 134. In this manner, the reel assembly 104, which in the illustrated embodiment is coupled with head cushion 134, may be operated to tension both the head cushion 134 and body portion 102. In some embodiments, the tubing 108 may couple with one or more straps 116 that in turn couple with body portion 102 or head cushion 134 via buckles 110.

In other embodiments, the arm portions 109 may include lace guides 107 that direct and guide the lace 108 along a lace path as described herein. The lace guides 107 may include a channel or lumen within which the lace 108 is positioned. The lace guides 107 may be coupled with the arm portions 109 (or with body portion 102 or had restrained 134) via buckles 110. In such embodiments, the tubing 120 may be positioned along body portion 102 and/or head cushion 134. Such a configuration allows the reel assembly 104 to simultaneously tension an upper arm portion 109 and a lower arm portion 109 of restraint member 106. For example, the lace 108 may extend from reel assembly 104 to a first lace guide 107 positioned on an upper arm portion 109. The lace 108 may then be guided along the body portion 102 via tubing 120 and to a second lace guide 107 positioned on a lower arm portion 109. The lace 108 may then be directed across a bottom portion of body 102 and to lace guides (not shown) positioned on opposite side of mask 100. The lace may then return to reel assembly 104 and/or terminate on the mask 100. The reel assembly 104 may be operated to tension lace 108 and thereby tightening the mask 100 about the user's face.

Reel assembly 104, along with the other reel assemblies described herein, typically includes a knob that may be grasped and rotated by a user to wind lace 108 about a spool that is positioned within a housing of reel assembly 104, or to unwind lace 108 therefrom. As the lace 108 is wound about the spool, the lace 108 is tensioned, which pulls the mask 100 against the user's had and/or face. In some embodiments, the lace 108 may pull on straps or other components of body 102 and/or head cushion 134 to enable tightening of the mask 100. Exemplary emodiments of reel assemblies are described in the following U.S. Patent Applications, the entire disclosures of which are incorporated by reference herein: U.S. patent applications Ser. No. 13/343,658, filed Jan. 4, 2012, entitled “Reel Based Closure System,” U.S. patent applications Ser. No. 13/273,060, filed Oct. 13, 2011, entitled “Reel-Based Lacing System,” and U.S. patent applications Ser. No. 13/098,276, filed Apr. 29, 2011, entitled “Reel Based Lacing System.”

An advantage of mask 100 compared with conventional masks is that the single reel assembly 104 may be used to tighten the body 102 and head cushion 134 about the user's head and face. Further, the fit or tightness of the mask 100 is roughly uniform due to the use of lace 108, which is used to tighten both the upper and lower portions of the mask 100 (i.e., the head cushion 134 and body 102). Uniform fit and/or tightness is provided because the tension within the lace 108 is roughly uniform. Another advantage of mask 100 is that a user may adjust the tightness and/or fit of the mask via reel assembly 104 without opening or uncoupling straps of the mask, which is often required with conventional masks. Rather, the user may easily don the mask 100 via the use of buckles 110 and then make all subsequent adjustments via reel assembly 104. Reel assembly 104 additionally allows micro-adjustments (i.e., in mm increments) of the fit or tightness of the mask to be made.

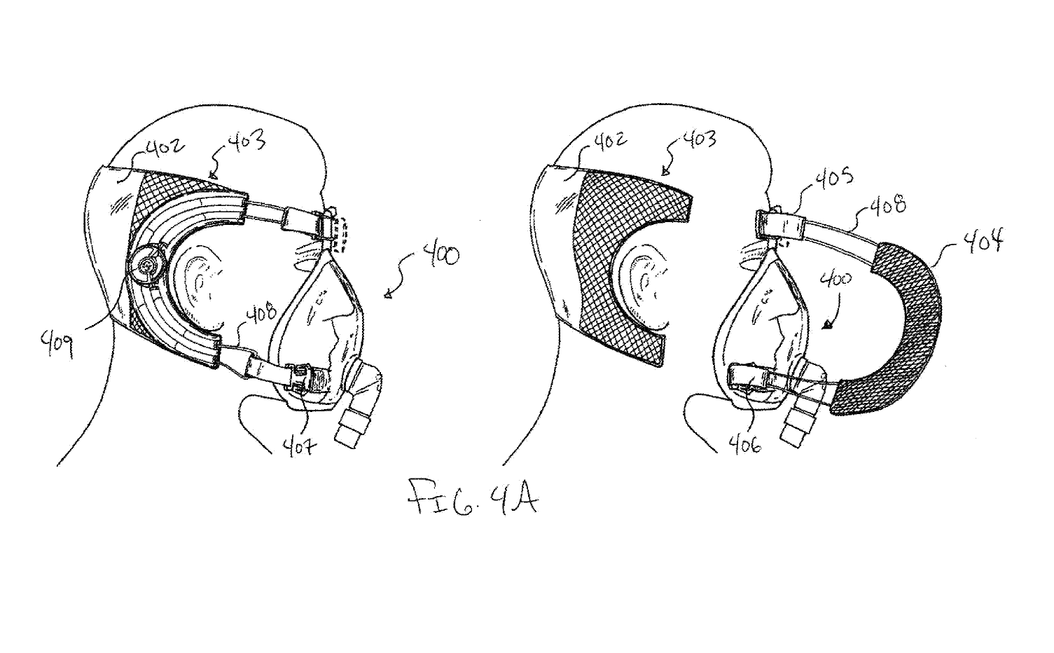

In exemplary embodiments, one or more of the buckles 110 may include a male coupling component (see buckle 425 of FIG. 4C) that releasably couples with a female coupling component (see component 426 of FIG. 4C) of the mask or other component. When coupled together, the male coupling component and female coupling component may have a relatively thin and flat profile that provides a sleek appearance and/or reduces contact with surrounding objects. Furhter, the male coupling component and female coupling component may not be removable or uncouplable while under tension. As such, accidental opening of the mask may be avoided, such as due to accidentally contacting the buckle 110 via moving or shifting during sleep and the like. Exemplary embodiments of male coupling components and female coupling components are further described in the '435 application, which is incorporated by reference herein. In exemplary embodiments, any of the buckles described herein may be replaced by male and female coupling components.

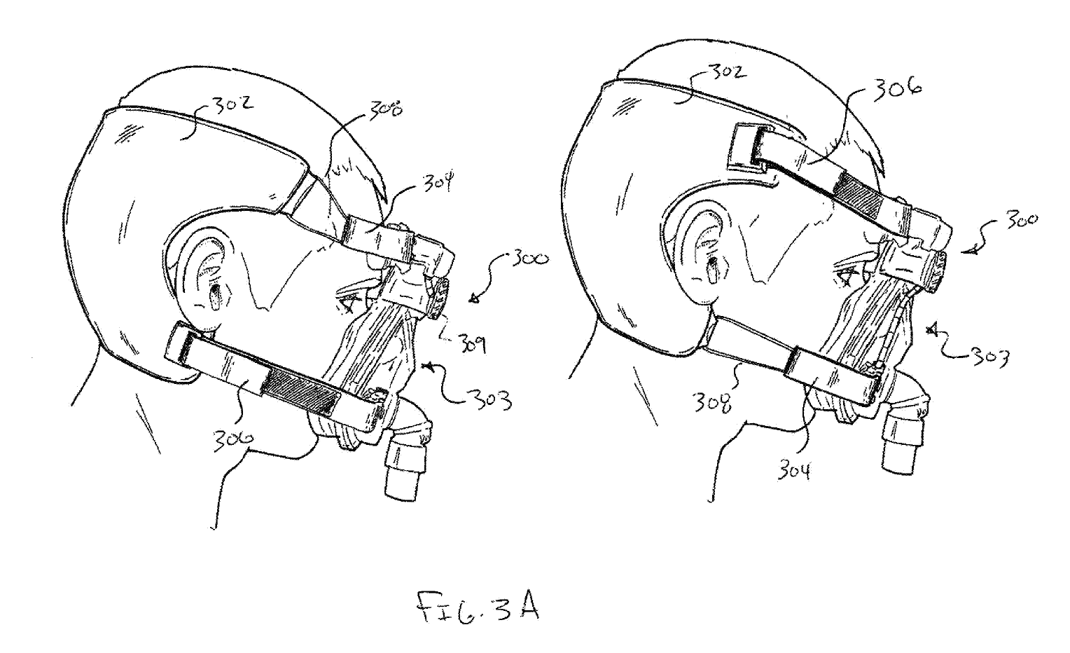

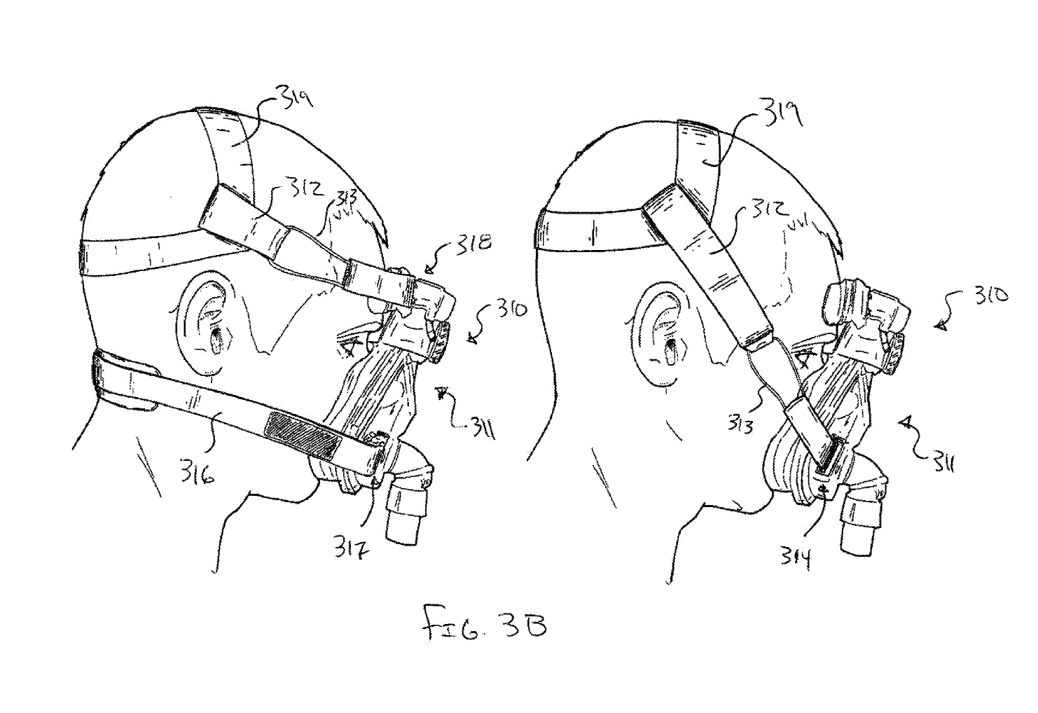

Referring now to FIG. 2, illustrated is an embodiment of a mask 200 fit about a user's head. Mask 200 includes a body 202 and a head cushion that is positioned above body 202. A reel assembly 204 is coupled with the head cushion and may be used to tension lace 208 which is coupled with a restraint member 206 via tubing 222 and/or lace guides (not shown). The mask 200 may be easily donned and doffed via buckles 210 as described herein. FIG. 2 shows that the use of the lace 208 allows the mask 200 to be dynamically fit about the user's head. For example, the restraint member 206 may be moved about the user's head so as to provide maximum comfort to the user. As the restraint member 206 is moved about user's head, the lace 208 moves or shifts within the tubing 220 and/or lace guides to maintain a uniform and/or consistent fit/tightness of the mask 200 about the user's head and face. Further, as the body 202 and/or head cushion shift during use of the mask 200, the lace 208 is able to shift within the tubing and/or lace guides to maintain the uniform and/or consistent fit/tightness of the mask 200 about the user's head. The lace 208 similarly moves or shifts within the tubing 220 and/or lace guides as the reel assembly 204 is operated to tighten the mask 200. In this manner, the fit of the mask 200 is dynamic and able to compensate for movement and/or shifting of the mask components. Conventional masks are unable to compensate for such movement and/or shifting.

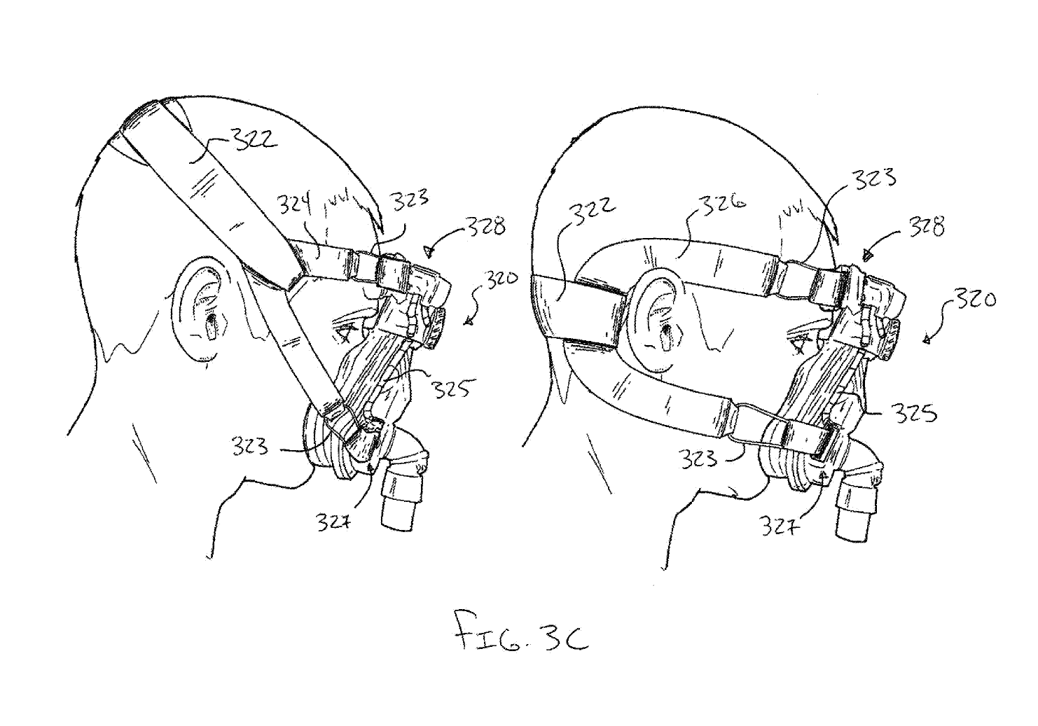

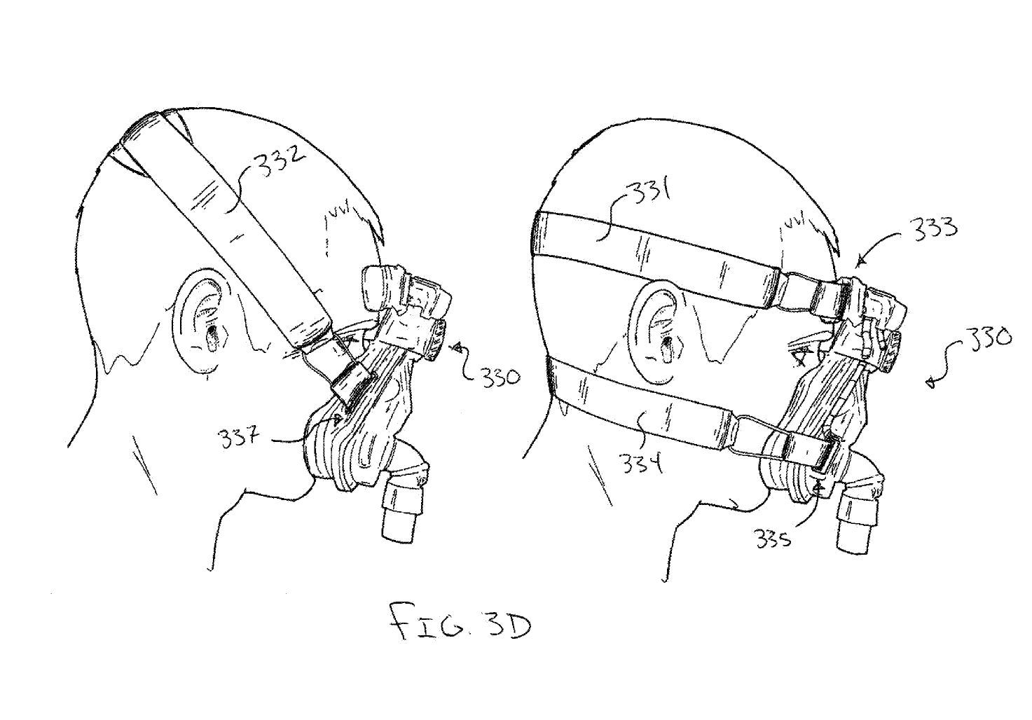

Referring now to FIGS. 3A-D, illustrated are various embodiments of straps that may be used to couple a mask with a user's head. Specifically, FIG. 3A illustrates a mask 300 having a restraint member 302 that couples with a main body 303 of the mask 300. As shown in the left-hand image, the head cushion, or upper portion of the main body 303, couples with restraint member 302 via lace 308 and a first strap 304. The lace 308 is coupled with the reel assembly 309 to allow the upper portion of main body 303 to be tensioned via the lace. The lower portion of main body 303 is coupled with the restraint member 302 via a second strap 306, which in the illustrated embodiment includes a Velcro® attachment mechanism although other attachment mechanisms may be used. The right-hand image of FIG. 3A illustrates an opposite configuration, in which the lace 308 and first strap 304 are coupled with a lower portion of main body 303 and the second strap 306 is coupled with the upper portion of main body 303. The embodiment of FIG. 3A allows for either the upper or lower portion of main body 303 to be tightened via the reel assembly and lace 308. Such embodiments may be preferred when only a single portion or zone of the mask 300 needs to be tightened, such as when the tightness of the lower portion of main body 303 needs to be tightened to minimize or eliminate any gaps between the user's face and cushioning of the main body 303.

The left-hand portion of FIG. 3B illustrates the use of two separate straps rather than the use of a restraint member. In the illustrated embodiment, a first strap 312 is coupled with an upper portion 318 of main body 311 via lace 313, which in turn is coupled with, and tensionable by, a reel assembly. The distal end of the first strap 312 is coupled with an additional strap 319 that wraps around the crown of the user's head. The first strap 312 is tensionable by tensioning the lace 313 via the reel assembly. A second strap 316 is coupled with a bottom portion of main body 311. A proximal end of the second strap 316 may include a buckle 317 that may be coupled with main body 311, such as by inserting the buckle 317 within a corresponding slot of main body 311. The first strap 312 and second strap 316 are independently tightenable such that the upper and lower portions of main body 311 may be independently tightened about the user's face to vary the fit in the upper and lower portions of the main body 311.

The right-hand image of FIG. 3B illustrates the use of a single strap 312 for coupling the mask 310 to the user's face. As previously described, the single strap 312 is coupled at a distal end with another strap 319 which wraps around a crown of the user's head. The proximal end of the single strap 312 is coupled with a buckle 314, which is inserted within a slot of main body 311. The single strap 312 in turn is coupled with lace 313 that is tensionable via a reel assembly of mask 310. The use of the single strap 312 allows the mask 310 to be easily donned and doffed by simply placing the single strap 312 about the user's head.

The left-hand image of FIG. 3C illustrates a single strap 322 which is wrapped around a crown of the user's head and that couples at a proximal end with a V-shaped strap 324 that attaches to the upper portion 328 of mask 320 and a lower portion 327 of mask 320. The upper and lower portions of the V-shaped strap 324 are coupled with lace 323, which in turn is coupled with a reel assembly. The reel assembly may be operated to tension the lace 323 and thereby tension the V-shaped strap 324 and single strap 322 about the user's head and thereby pull or tighten the mask 320 about the user's face. Tubing 325 is coupled with the mask 320 and routes the lace 323 between the upper portion 328 and lower portion 327 of mask 320. The use of the V-shaped strap 324 allows the single strap 322 to be positioned above the user's ear.

The right-hand image of FIG. 3C also illustrates the use of a single strap 322, but the single strap 322 is wrapped around a base of the user's head. A proximal end of the single strap 322 is coupled with a U-shaped strap 326 that fits or is positioned around the user's ear. The U-shaped strap 326 in turn is coupled with the upper portion 328 and lower portion 327 of mask 320 via lace 323, which is coupled with the mask's reel assembly. As described above, the reel assembly may be operated to tension the lace 323 and thereby tension the U-shaped strap 326 and single strap 322 about the user's head, and thereby pull or tighten the mask 320 about the user's face. Tubing 325 is coupled with the mask 320 and routes the lace 323 between the upper portion 328 and lower portion 327 of mask 320. The use of the V-shaped strap 324 and U-shaped strap 326 allows the position of the single strap 322 to be adjusted about the user's head for comfort and/or other purposes.

The left-hand image of FIG. 3D is similar to the right-hand image of FIG. 3B in that a single strap 332 is wrapped about the crown of the user's head. The proximal end of the single strap 332 is coupled roughly centrally 337 with mask 330. The proximal end of the single strap 332 is also coupled with lace (not numbered) that is in turn coupled with a reel assembly of the mask for tensioning or tightening the mask about the user's face as described herein. The difference between the left-hand image of FIG. 3D and the right-hand image of FIG. 3B is that the single strap 332 is not coupled at a distal end with an additional strap that is wrapped around the user's head. Rather the single strap 332 is used alone to couple the mask 330 about the user's head.

The right-hand image of FIG. 3D illustrates the use of two straps for coupling the mask 330 about the user's head. Specifically, a first strap 331 is coupled with an upper portion 333 of mask 330 while a second strap 334 is coupled with a lower portion 335 of mask 330. The first strap 331 and/or second strap 334 may each be coupled with lace (not numbered) that is tensionable by a reel assembly of mask 330. Tubing (not numbered) may be used to route the lace about the mask 330 between the upper portion 333 and lower portion 335. Although not specifically described, any of the straps illustrated in FIGS. 3A-D may include buckles or other attachment components (e.g., the male and female coupling components) for coupling the straps with the respective masks.

Referring now to FIGS. 4A-D, illustrated are embodiments of attachment mechanisms for attaching the mask/head restraints to an individual's head. The attachment mechanisms illustrated in FIGS. 4A-D represent gross or macro closure mechanisms that may be used to initially close the mask. Further closure or tensioning of the mask may then be performed with the reel assembly and lacing system as described herein.

FIG. 4A illustrates an embodiment in which a mask 400 is coupled with a restraint member 402 via a removable portion 404. To couple the removable portion 404 with the restraint member 402, the restraint member 402 includes a coupling portion 403 that is configured to couple with the removable portion 404. In some embodiments, the coupling portion 403 of restraint member 402 and the removable portion 404 includes corresponding hook and loop fastening components such as those commonly employed in Velcro® straps. In coupling the two components, the mask 400 may be placed about the user's face and the restraint member 402 may be wrapped around the user's head. The removable portion 404 may then be folded over a side of the user's face and coupled with a coupling portion 403.

In some embodiments, the removable portion 404 may be generally U-shaped and proximal ends of the removable portion 404 may be coupled with the mask 400. For example, an upper portion of the removable portion 404 may be coupled with an upper portion 405 of mask 400 while a lower portion of removable portion 404 is coupled with a lower portion 406 of mask 400. The upper and lower portions of removable portion 404 may also include straps that are attached to lace 408, which is in turn coupled with the reel assembly of mask 400. In some embodiments one or more buckles 407 may be used to couple the upper and lower portions of removable portion 404 with the upper and lower portions, 405 and 406 respectively, of mask 400. The buckles 407 may be conventional buckles or, in exemplary embodiments, may include male and female coupling components as described in the '435 patent application incorporated herein.

In some embodiments, the reel assembly 409 is positioned on the removable portion 404. For example, the left-hand image of FIG. 4A illustrates the reel assembly 409 positioned roughly centrally about removable portion 404. Tubing (not numbered) and/or lace guides (not shown) may be used to route the lace 408 to and from the reel assembly 409. As described herein, operation of the reel assembly 409 tensions lace 408, which in turn tensions or tightness the various components of mask 400 about the face. In other embodiments, the reel assembly may be attached to the mask 400 as shown by the reel assembly in dashed lines.

Referring now to FIG. 4B, illustrated is another embodiment of attaching a mask 410 about a user's face. As with the previous embodiments, mask 410 includes a reel assembly 416 that is operable to tension lace 415 and thereby adjust the fit or tightness of the mask 410 about the user's face. Tubing 417 may be positioned about mask 410 to route the lace 415 along the mask 410 and to and from one or more lace guides 413. The mask 410 is couplable with a first strap 412 and a second strap 414, which each include a lace guide 413 at a proximal end thereof. The first and second straps, 412 and 414, are each positionable around the head as described herein.

The lace guides 413 are releasable guides that have an open back or rear surface around or about which the lace 415 may be positioned. The open rear surface of the lace guides 413 forms a channel within which the lace 415 is positioned to couple the respective guides 413 and respective straps with the mask 410. For example, a loop formed in the lace 415 may be pulled over a lace guide 413 and positioned within the channel of the lace guide's rear surface. The lace 415 may then be tensioned via reel assembly 416 to secure the lace 415 within the channel of the lace guide 413 as shown in the right-hand image of FIG. 4B. The open rear surface configuration of the lace guides 413 allows the lace 415 to be easily and quickly coupled and uncoupled with the lace guides 413 in donning and doffing of the mask 410.

In some embodiments, only a single side of the mask 410 may include the lace guides 413. In such embodiments, an opposite side of the mask 410 may be permanently attached or coupled with the first strap 412 and the second strap 414. In such embodiments, a side of the mask 410 that includes the lace guides 413 may be used for donning and doffing of the mask 410. In other embodiments, both sides of the mask 410 may include the lace guides 413 so that the mask 410 may be fully removed from the first strap 412 and the second strap 414. Fully removing of the mask 410 from the first strap 412 and the second strap 414 may allow the mask 410 and/or first strap 412 and second strap 414 to be removed and replaced without requiring replacement of the entire mask assembly.

FIG. 4C illustrates an embodiment in which the male and female coupling components of the '022 patent application are used for attaching a restraint member 422 with a mask 420. Specifically, as illustrated a male coupling component 425 having a stepped protrusion as described in the '435 patent application is inserted within a corresponding recess of a female coupling component 426 that is formed into a body of mask 420, or otherwise coupled therewith. A distal end of the male coupling component 425 is coupled with lace 428 that is in turn operationally coupled with a reel assembly 424. The proximal end of the male coupling component 425 may include a lumen or channel within which the lace 428 is positioned such that the proximal end of the male coupling component 425 functions as a guide for the lace 428.

When coupled with the female coupling component 426, the male coupling component 425 is not removable or uncouplable from the female coupling component 426 while the lace 428 remains under tension. As such, accidental opening of the mask 420 is prevented. Further, when coupled together, the male coupling component 425 and the female coupling component 426 have a flat profile that may match the shape and/or curvature of mask 420. As illustrated, an upper portion of the mask 420 may be permanently coupled with restraint member 422 via lace 428. This configuration allows the reel assembly 424 to be positioned on mask 420.

FIG. 4C also illustrates another embodiment of a mask 430 that is coupled with a restraint member 432 via a plurality of male coupling components 433. As described herein, the male coupling components 433 are insertable within corresponding female coupling components 435 of mask 430. In the illustrated embodiment, the reel assembly 434 is positioned on an upper male coupling component 433. The reel assembly 434 is attached with lace 438 which is routed through restraint member 432 and to the lower male coupling component 433. In such an embodiment, the reel assembly 434 is not permanently coupled with the mask 430. Such configurations allow the mask 430 (i.e., the hard good) to be removed from the restraint member 432 (i.e., the soft good) and replaced with another mask. Such configurations also provide the advantage of allowing the user to grasp the reel assembly 434 to couple the upper male coupling component 433 and then to subsequently tension the lace 438 via the reel assembly 434. These operations (i.e., coupling of component 433 and tensioning of lace 428) may often be performed with a single hand since the reel assembly 434 is combined with the male coupling component 433. Further, the user does not have to reposition their hand from the male coupling component 433 in order to tension the lace 438.

In some embodiments, only a single side of the mask 430 includes the male coupling components 433. In such embodiments, an opposite side of the mask 430 may be permanently attached or coupled with the restraint member 432. In such embodiments, the side of the mask 430 that includes the male coupling components 433 may be used for donning and doffing of the mask 430. In other embodiments, both sides of the mask 430 include the male coupling components 433 so that the mask 430 is fully removable from the restraint member 432. In some embodiments, one or more components of the closure device (e.g., the male coupling components 433, reel assembly 424, and the like) may be attached to a common backing, such as a rigid, semi-rigid, or soft panel. The user may grasp the panel and couple the male coupling component(s) 433 with the female coupling component(s) 435 of the mask and/or operate the reel assembly 424. The use of panels may aid in donning and doffing of the mask and/or tensioning of the mask. Exemplary embodiments of panels that may be used with the mask are described in U.S. Provisional application Ser. No. 14/173,685, filed Feb. 5, 2014, and titled “Closure Devices for Medical Devices and Methods”, the entire disclosure of which is incorporated by reference herein.

FIG. 4D illustrate another embodiment in which a mask 448 (i.e., the hard good) is fully removable from a restraint member 442 (i.e., the soft good). The restraint member 442 includes a plurality of attachment components that are each positioned at a distal ends of arm portions 445. In the illustrated embodiment, male coupling components 443a are positioned at the distal end of upper arm portions 445a while conventional buckles 443b are positioned at the distal end of lower arm portions 445b. Mask 440 includes corresponding female coupling components 444a that are positioned on an upper portion of the mask's body and also includes conventional buckles slots 444b that are positioned on a lower portion of the mask's body. The male coupling components 443a are insertable within the female coupling components 444a while the conventional buckles 443b are insertable within the conventional buckles slots 444b. Inserting the attachment components of restraint member 442 into the corresponding slots of mask 440 couples the restraint member 442 with the mask 440.

A proximal end of the arm portions 445 is coupled with lace 447, which is routed through a lumen or channel of restraint member 442. The lace 447 allows the upper and lower arm portions, 445a and 445b, to move or shift dynamically relative to restraint member 442 as described herein. A distal end of the upper arm portions 445a is coupled with second lace 449, which is in turn coupled with reel assembly 446. Reel assembly 446 is positioned roughly centrally between opposing upper arm portions 445a of restraint member 442. Reel assembly 446 is operable to tension the second lace 449, which in turn tensions upper arm portions 445a. Tensioning of the upper arm portions 445a tensions the lower arm portions 445b via lace 447.

Reel assembly 446 is configured for coupling with an attachment site 441 of the upper portion of mask 440. In some embodiments, the reel assembly 446 is attached with a strap 448 which is positionable around the attachment site 441 of mask 440. The strap 448 may include Velcro® components that allow the strap 448 to be attached around attachment site 441. In other embodiments, other attachment mechanisms may be used to attach strap 448 or reel assembly 446 with attachment site 441. For example various mechanical fasteners, such as snaps, clamps, cams, locks, pins, and the like may be used to attach strap 448 or reel assembly 446 with attachment site 401. The embodiment of FIG. 4D allows the soft goods (i.e., restraint member 442) to be uncoupled and removed from the hard goods (i.e., mask 440), such as for replacement, cleaning, inspection, and the like.

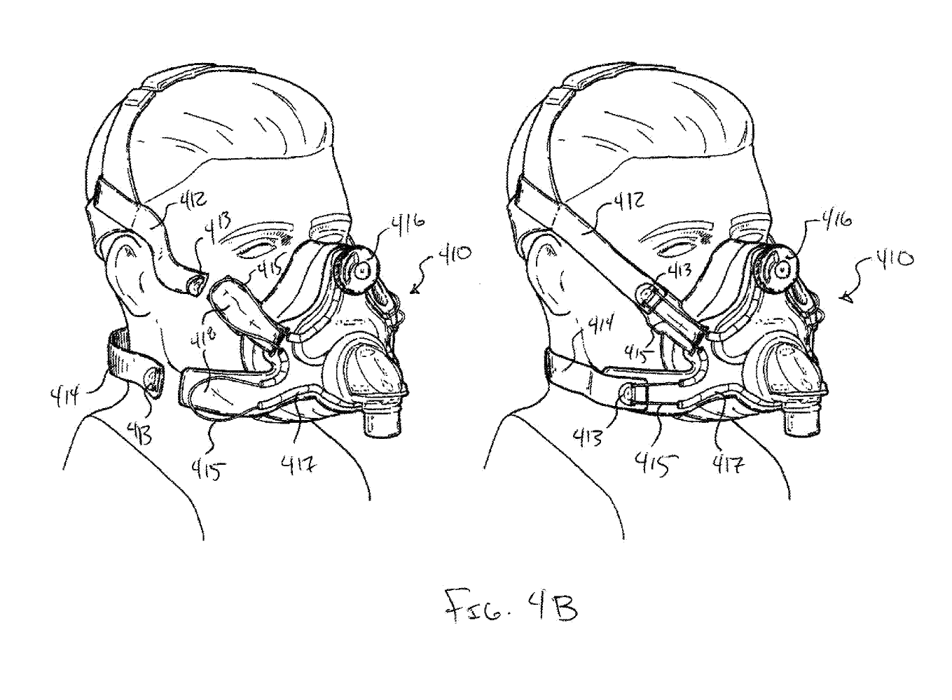

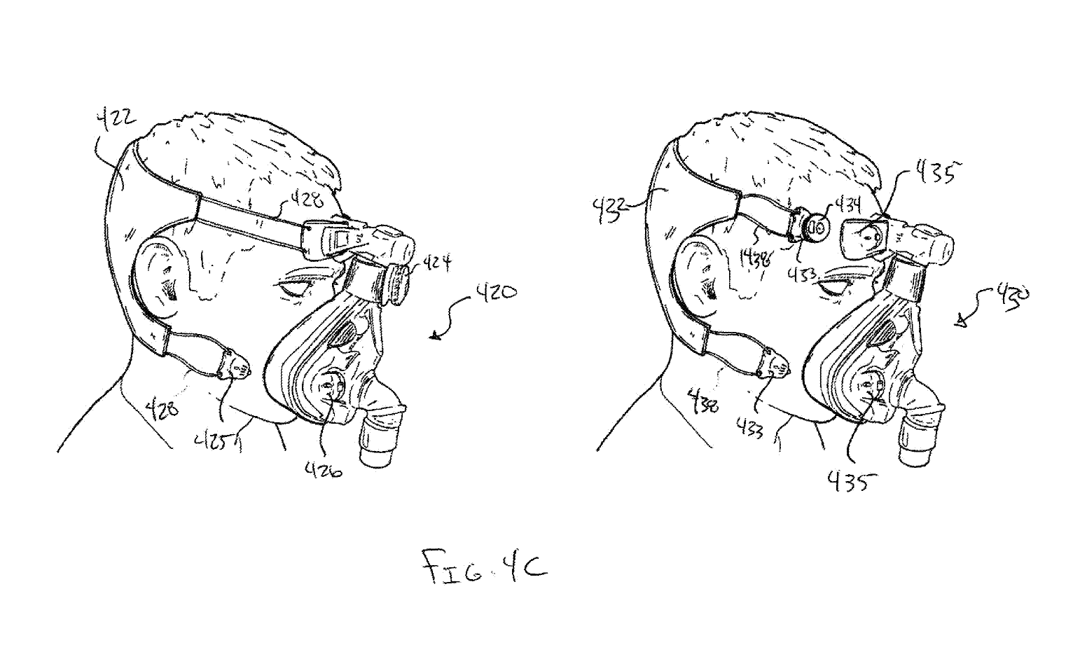

Referring now to FIGS. 5A-C, illustrated are embodiments that show various positioning of the reel assembly about a mask/head restraint. The position of the reel assembly relative to the mask may be adjusted based on a desired closure or fit of the mask and/or based on access. For example, positioning of the reel assembly near the front of the mask may provide a user with easy access to the reel assembly and/or provide increased comfort when sleeping or resting. Positioning of the reel assembly near a rear surface of the mask assembly may provide a physician or technician who may be fitting the product with easy access to the reel assembly.

The left-hand image of FIG. 5A illustrates a mask 500 that is coupled with a restraint member 502 via lace 508 and tubing 506 and/or one or more buckles 509 as described herein. The restraint member 502 is configured similar to a helmet that is fit about the user's head. The reel assembly 504 is coupled with the restraint member 502 near or toward mask 500. The positioning of the reel assembly 504 in this manner allows the reel assembly 504 to be easily accessed by the user. The right-hand image of FIG. 5A illustrates a similar mask 500 and restraint member 502 configuration with the reel assembly 504 positioned near the top of restraint member 502 and away from mask 500. The positioning of the reel assembly 504 in this configuration allows the reel assembly 504 to be easily accessed by a physician or technician that may be initially fitting the mask 500 about the user's face. The lace 508 may be inserted through tubing 506 from the reel assembly 504 and to mask 500.

FIG. 5B illustrates a similar embodiment of a mask 510 and restraint member 512 that are coupled via lace 518 and a plurality of tubing 516. Restraint member 512 is positioned around the back of the user's head. The reel assembly 514 is positioned on a rear surface of restraint member 512 opposite mask 510. The lace 518 is routed to and from reel assembly 514 via tubing 516. The restraint member 512 includes compressible material 513 that covers, conceals, and/or hides the reel assembly 514 from view and/or protects the reel assembly 514 from accidental contact with nearby objects, which contact may otherwise cause the reel assembly 514 to accidentally open or loosen the lace tension. The compressible material 513 may be pushed downward to allow the user to access reel assembly 514 and thereby tension lace 518 and tighten mask 510. Exemplary embodiments of compressible materials 513 are further described in U.S. patent application Ser. No. 13/829, 601, filed Mar. 14, 2013, entitled “Tightening Mechanisms and Applications Including the Same,” the entire disclosure of which is incorporated by reference herein.

FIG. 5C illustrates another embodiment of a mask 520 that is coupled with the restraint member 522 via lace 528 and/or one or more buckles 526. The reel assembly 524 of mask 520 is mounted roughly centrally about the body of mask 520. The lace 528 is routed to and from the reel assembly 524 via tubing 527 that is positioned atop the body of mask 520. Positioning the reel assembly 524 centrally about the mask 520 may provide the most easy access to the reel assembly for the user.

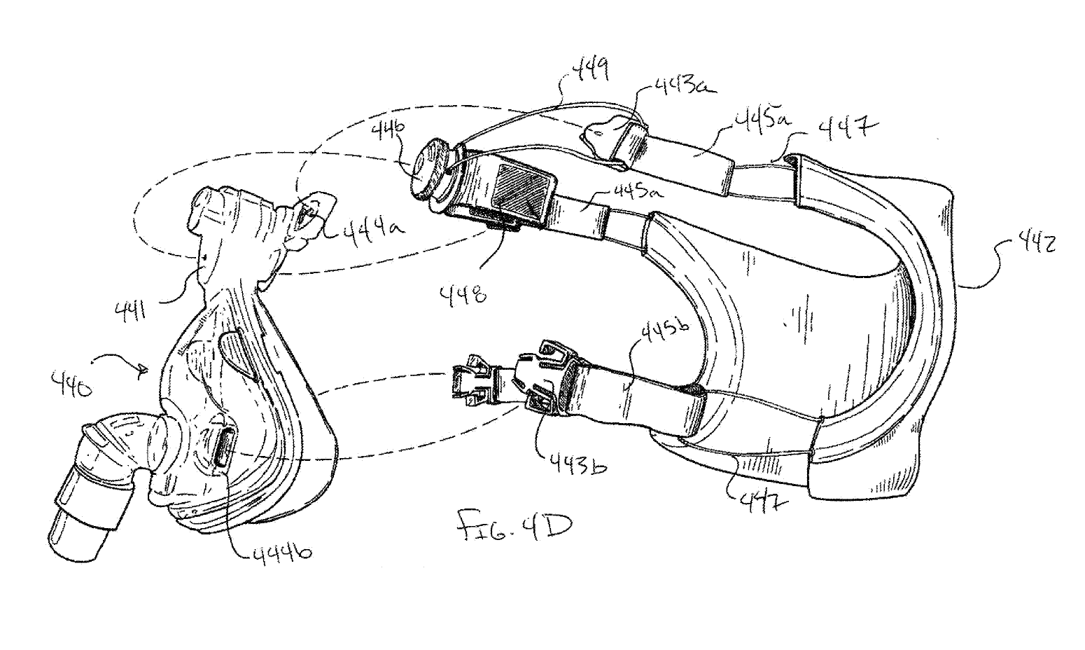

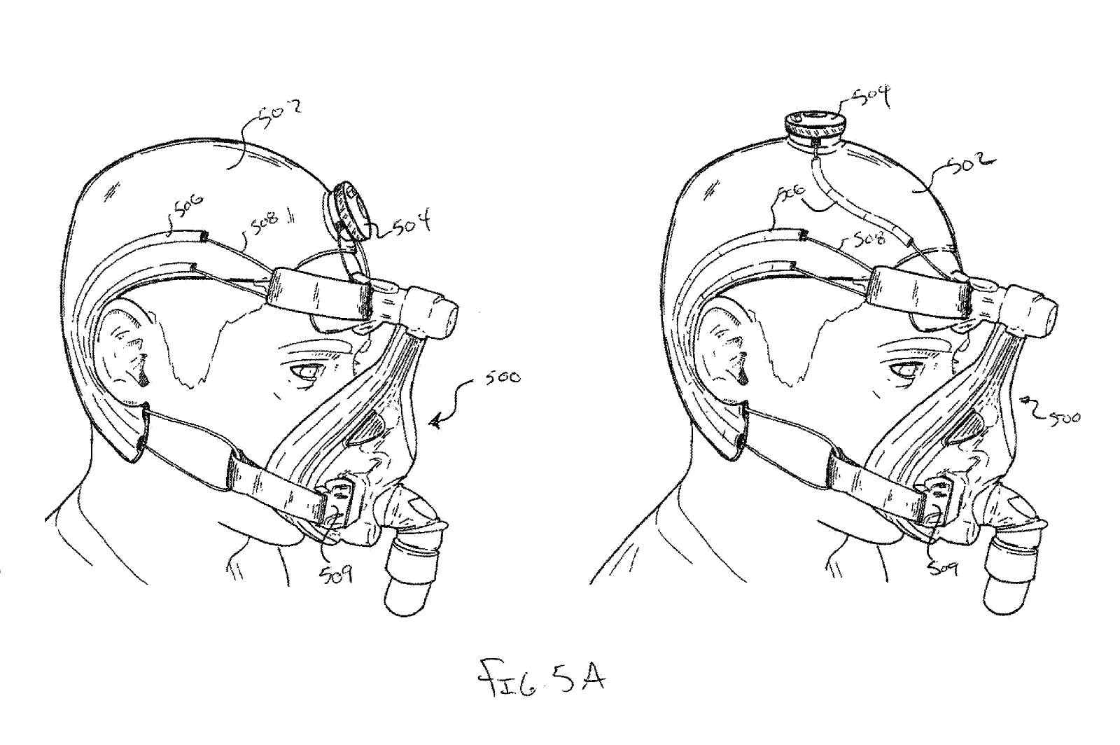

FIG. 6 illustrates an embodiment of a restraint member 608 that may be coupled with a conventional mask 600. Stated differently, the restraint member 608 is configured for coupling with a mask that is not necessarily designed for coupling with the restraint member 608. Coupling conventional masks 600 with the restraint member 608 may be desirable in order to allow the conventional masks 600 to be closed and tensioned via a reel assembly 620.

To couple the conventional masks 600 with the restraint member 608, the restraint member includes a front panel 610 and a rear panel 614. The front and rear panels, 610 and 614, arc attached or coupled together via lace 616 that is insertable through tubing 622 and/or one or more guides 624 attached to the front and rear panels, 610 and 614. In some embodiments, male and female coupling components, such as those described in the '435 patent application incorporated herein, may be used to couple the front and rear panels, 610 and 614.

The rear panel 614 is configured for positioning about the rear surface of the user's head. The front panel 610 may include a slot or opening 612 through which the inlet port 602 of the conventional mask 600 is positioned. The front panel 610 is configured for positioning over the conventional mask 600. In some embodiments, both the front panel 610 and the rear panel 614 are made of relatively soft and compliant fabrics or other materials that allow the front and rear panels, 610 and 614, to conform to the rear surface of the user's head and to the conventional masks 600 respectively. With the rear panel 614 positioned about the user's head, the reel assembly 620 may be operated to wind the lace 616 about the reel assembly's spool and thereby move the front panel 610 toward the rear panel 614 and into contact with conventional mask 600. The reel assembly 620 may be operated until the front panel 610 fully contacts the conventional mask 600 and/or until a desired fit or tightness of the mask 600 about the user's face is achieved.

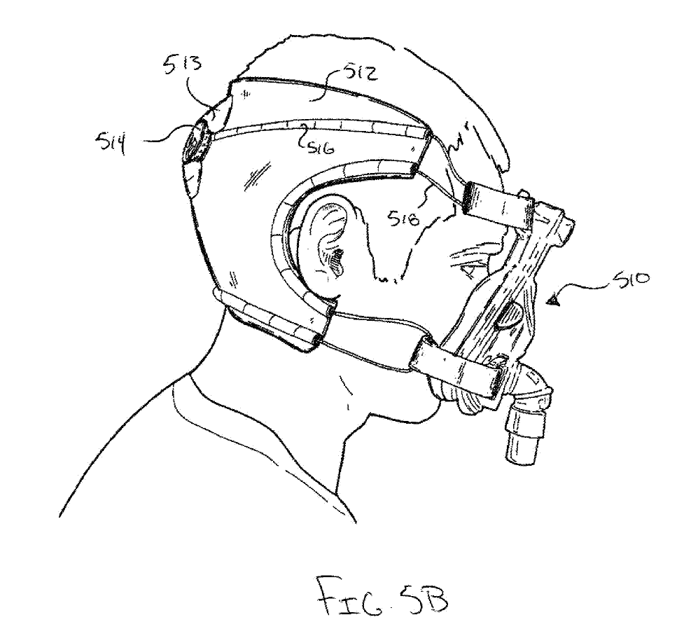

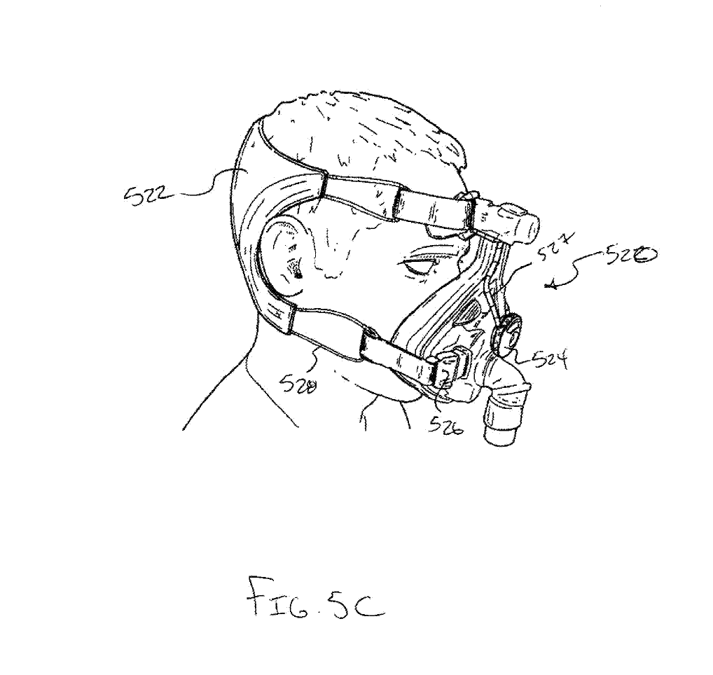

Referring now to FIG. 7, illustrated is an embodiment in which the restraint member or rear panel 702 of a mask assembly is thermoformable to a rear surface of the user's head. To custom form the rear panel or restraint member 702 to the user's head, the rear panel 702 is softened by heating the rear panel 702 and then the rear panel 702 is pressed against the rear surface of the user's head. The rear panel 702 is then cooled to harden the rear panel 702 in the shape of the user's head. An inner surface of the rear panel 702 that contacts the user's head may include soft fabrics or cushioning so as to increase the comfort of wearing the rear panel 702 and mask assembly. The rear panel 702 may include one or more lace guides (not shown) and/or tubing (not shown) that couple with lace (not shown) to attach the rear panel 702 with mask 700. In some embodiments, the lace guides (not shown) may be releasable guides as described herein to allow the lace (not shown), and thereby the mask 700, to be easily coupled with rear panel 702. As described herein, the lace is operationally coupled and tensionable by reel assembly 708. The lace may be coupled with one or more straps 704 that may be releasably coupled with mask 700 via one or more buckles 706.

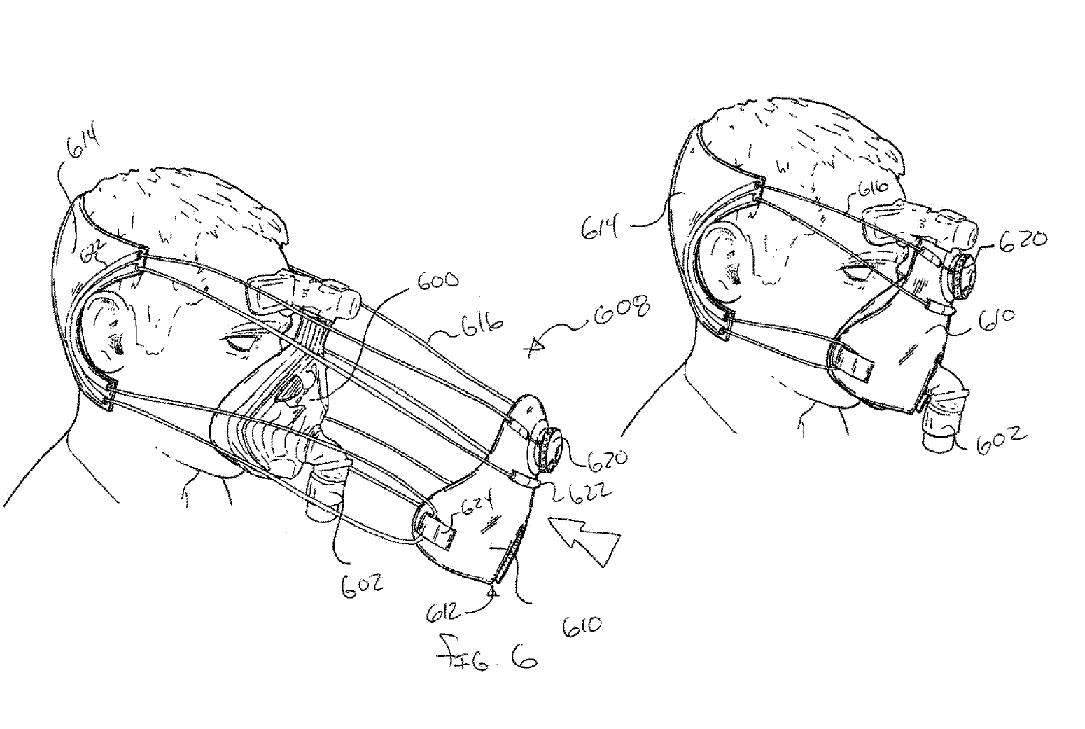

Referring now to FIG. 8, illustrated is an embodiment of a mask 800 that includes an attachment port 802 that is configured for releasably coupling with a reel assembly 808. Specifically, reel assembly 808 is attached to a housing 804 that includes a plurality of attachment components 806 that couple with the port 802 of mask 800. In some embodiments, the attachment components 806 may be flanges or other members that extend radially from the housing 804 of reel assembly 808. The flanges 806 may be insertable within slots (not shown) of attachment port 802. The flanges 806 may snap into engagement with the slots of attachment port 802. In some embodiments, the attachment port 802 further includes a release post 805 that may be pressed to uncouple the flanges 806 from the slots of the attachment port 802 and thereby allow the reel assembly 808 to be removed from the attachment port 802. Detaching the reel assembly 808 from mask 800 may be performed for replacement, inspection, and the like of reel assembly 808. In some embodiments, the reel assembly 808 may be removed and replaced with a higher or lower power reel assembly. Additional methods of attaching components of the closure device components to the masks/head restraints are described in U.S. Provisional Application No. 61/880,753, filed Sep. 20, 2013, entitled “Guides for Closure Devices and Methods Therefor”, the entire disclosure of which is incorporated by reference herein.

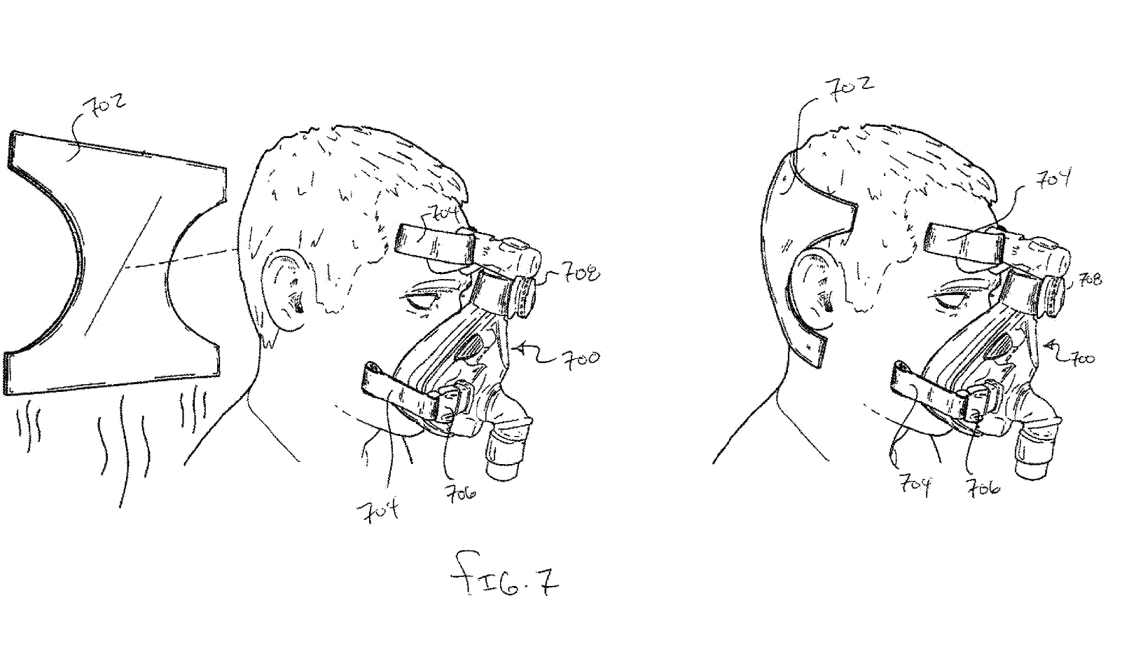

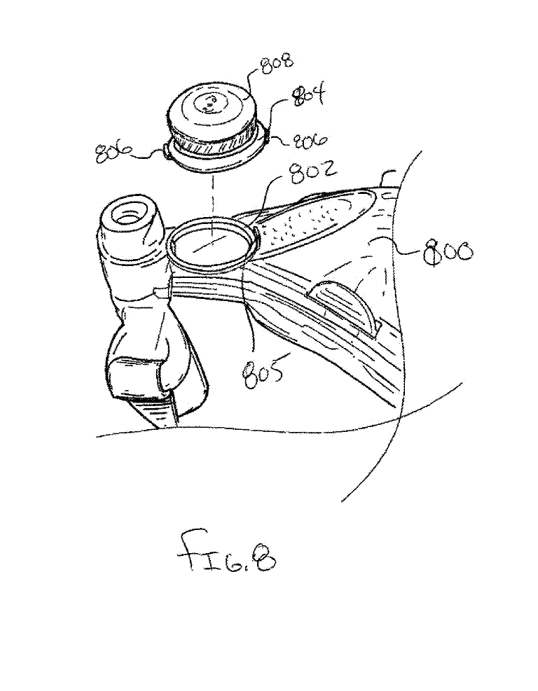

Referring now to FIG. 9, illustrated is an embodiment of a restraining member 902 that may be used to apply a perpendicular and roughly even pressure to the user's head. The restraining member 902 includes a plurality of pads 904 that are coupled with the lace 906 of the mask assembly 900 via lace guides 908. Each of the lace guides 908 includes a lumen or channel within which the lace 906 is positioned. The pads 904 are positioned within apertures of the restraint member 902 and are able to move radially inward and outward within the apertures and relative to restraint member 902. For example, as the lace 906 is tensioned via a reel assembly, the pads 904 are pressed radially inward within the apertures of restraint member 902 and against the user's head. Because the tabs 904 move radially inward and outward, the pressure exerted on the user's head from the tabs 904 is roughly perpendicular or directed radially inward. The pressure applied is also relatively uniform around and about the user's head, which may increase the comfort of wearing the mask assembly 900. In the illustrated embodiment, the pads 904 a roughly square or rectangle in shape, although various other shapes may be used.

The perpendicular force applied to the user's head via the pads 904 may be adjusted or varied by adjusting the height of the pad 904 relative to the restraint member 902. For example, in some embodiments, the pad 904a may be roughly equivalent in size, or slightly larger, than the restraint member 902. In such embodiments, the perpendicular force applied to the user's head may be relatively weak. In other embodiments, the pad 904b may be relatively large compared with the restraint member 902. In such embodiments, the perpendicular force applied to the user's head may be relatively strong. In varying the size of the pad 904, an outer surface of the pad may be offset a distance X from an outer surface of the restraint member 902. A larger distance X would result in a stronger perpendicular force being applied to the user's head, while a smaller distance X would result in a weaker perpendicular force being applied to the user's head.

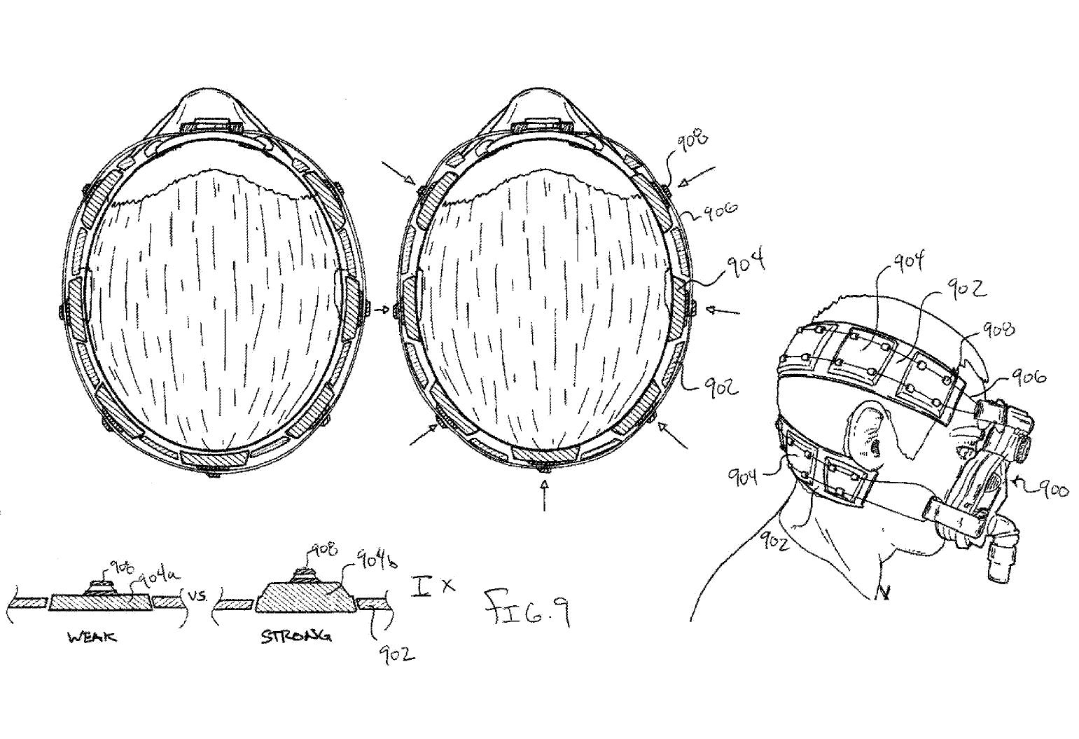

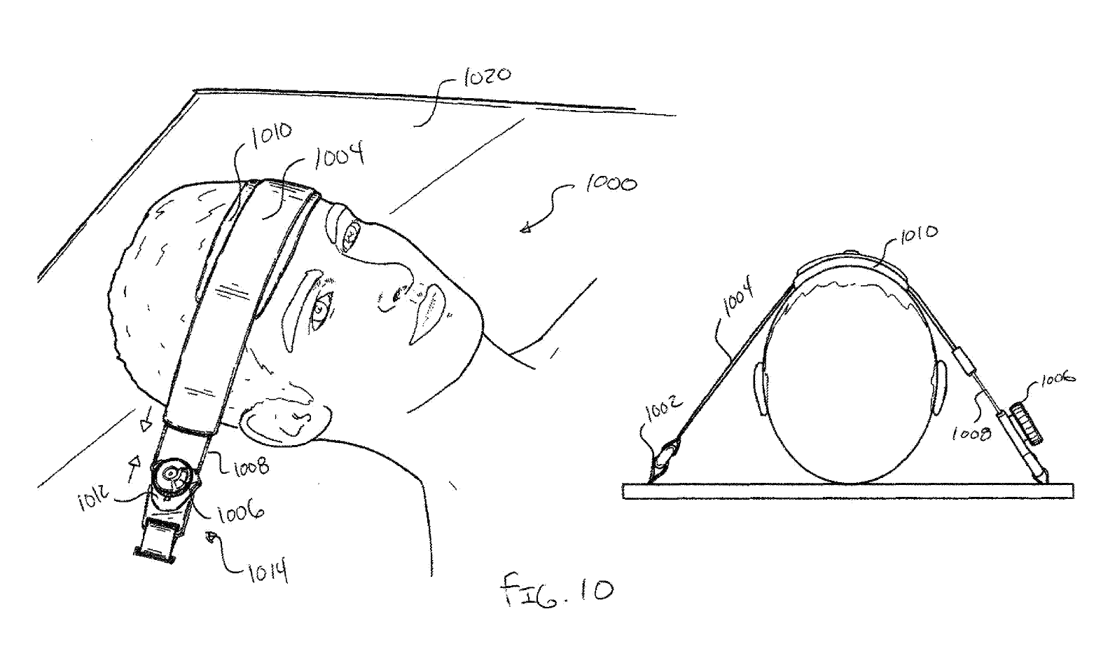

Referring now to FIG. 10, illustrated is an embodiment of a strap 1000 that may be used to restrain movement of a user's head. The strap 1000 may be coupled with a stretcher, operating or other table, or any other surface 1020 for which head restraint is desired. A proximal end 1002 of a strap body 1004 is permanently attached or coupled with the surface 1020 while a distal end 1014 is removably coupled with the surface 1020. The distal end 1014 of strap 1000 may include male and female coupling components 1012 as described in the '435 patent application incorporated herein, or may include any other attachment mechanism. An advantage of the male and female coupling components 1012 is that such components are not releasable or detachable while the strap body 1004 is tensioned. Another advantage of the male and female coupling components 1012 is that the components allow the distal end 1014 of the strap to be easily attached to the surface 1020 with a single hand. The single hand application of the male and female coupling components 1012 allows a technician (e.g., EMT, surgeon, and the like) to maintain a patient's head position with one hand and couple the male and female coupling components 1012 with the other. This process aids in spinal immobilization during field first aid as well as at the hospital.

The distal end 1014 of strap 1000 also includes a reel assembly 1006 that is operationally coupled with lace 1008 as described herein. The lace 1008 in turn is coupled with a distal end of strap body 1004. Tensioning of the lace 1008 draws or pulls the distal end of strap body 1004 toward reel assembly 1006 and thereby tensions or tightness strap body 1004. An inner surface of strap body 1004 may include a pad 1010 or other cushioning member that is pressed or pulled tightly against the user's head. In operation, strap body 1004 is positioned over the user's head and the male and female coupling components 1012 are coupled together with the strap body 1004 positioned over the user's head. The reel assembly 1006 may then be operated to tension the lace 1008 and thereby tension or tighten strap body 1004 about the user's head. The pad or cushion member 1010 provides comfort to the user's head as strap body 1004 is pulled tightly about the user's head. It should be realized that in other embodiments strap 1000 may be used to restrain other parts of the body, such as the hands, feet, torso, and the like.

In some embodiments, the reel assembly 1006 may have “incremental release” capabilities that allow the lace tension to be incrementally released or decreased by operation of the reel assembly 1006. For example, the reel assembly 1006 may be counter-rotated, or rotated in a loosening direction, to incrementally release or decrease the lace tension. Such incremental release technology allows a physician to release tension on the head for patient comfort and/or other purposes.

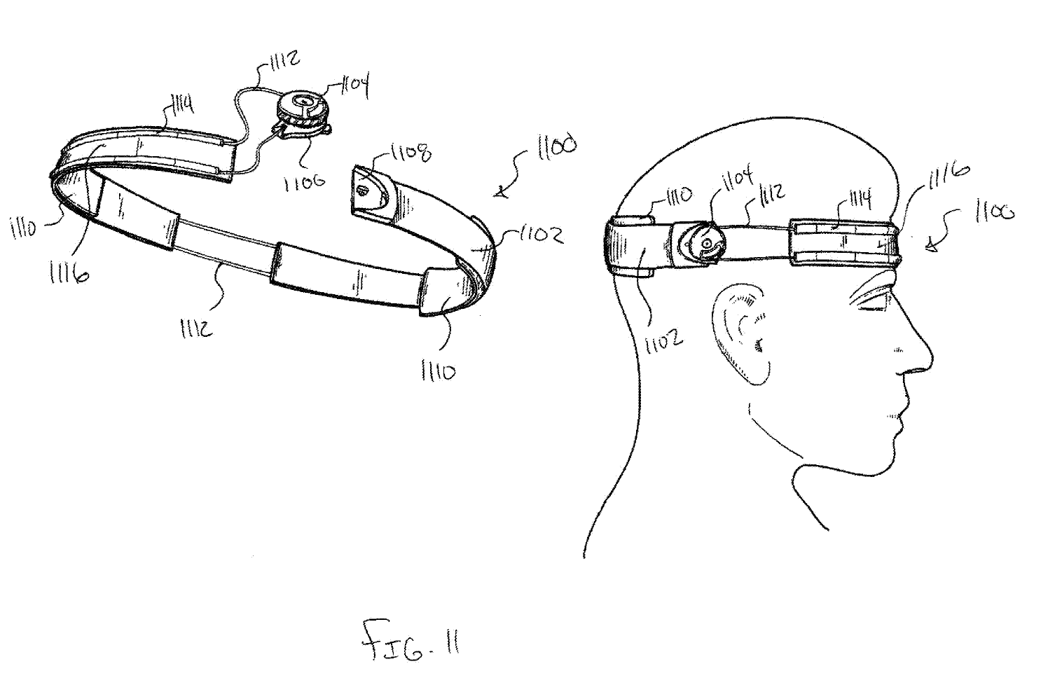

Referring now to FIG. 11, illustrated is a head restraint or band 1100 that may be fit and tensioned about a user's head. Head restraint 1100 includes a first strap portion 1102 and a second strap portion 1116 that are attached are coupled together via lace 1112. First strap portion 1102 includes a female coupling component 1108 that is couplable with a male coupling component 1106 as described in the '435 patent application incorporated herein. The head restraint 1100 may be placed about the user's head and the male and female coupling components, 1106 and 1108, coupled together to initially fit the head restraint 1100 about the user's head. The male coupling component 1106 includes a reel assembly 1104 that is operable to tension the lace 1112 and thereby tightening head restraint 1100 about the user's head. The lace 1112 may be inserted through tubing 1114 that is attached to either or both the first strap portion 1102 and second strap portion 1116. Operation of the reel assembly 1104 draws the first strap portion 1102 and second strap portion 1116 radially inward about the user's head. The first strap portion 1102 and/or second strap portion 1116 may include a pad or cushion member 1110 to provide comfort to the user as the head restraint 1100 is tightened about the user's head. It should be realized that in other embodiments restraint 1100 may be fit about other parts of the body, such as the hands, feet, torso, and the like.

What is claimed is:

1. A system for securing a mask about a user's face comprising:
a mask that is configured to fit about the user's face;
a padded member that is postionable on the back of the user's head;
at least one strap that extends from the padded member toward the mask; and
a closure system coupled with the mask and with the at least one strap, the closure system comprising:
a tension member;
a guide that routes the tension member about the mask and/or the at least one strap; and
a tensioning device that is operable to tension the tension member and thereby pull the at least one strap and padded member toward the mask to secure and/or tighten the mask about the user's face.

2. The closure system of claim 1, wherein the at least one strap is positioned on a first side of the user's head and wherein the system further comprises another strap that is positioned on an opposite side of the user's head.

3. The closure system of claim 1, wherein the tensioning device includes a rotatable knob.

4. The closure system of claim 1, wherein the tension member comprises lace.

5. The closure system of claim 1, wherein the tensioning device is positioned on the mask.

6. The closure system of claim 1, wherein the tensioning device is positioned on the padded member.

7. A method for securing a mask about a user's face comprising:
providing a mask having:
a mask member;
a padded member;
at least one strap that extends from the padded member toward the mask; and
a closure system coupled with the mask and with the at least one strap, the closure system comprising:
a tension member;
a guide that routes the tension member about the mask and/or the at least one strap; and
a tensioning device that is operable to tension the tension member;
positioning the mask member about the user's face;
positioning the padded member on the back of the user's head; and
operating the tensioning device to tension the tension member and thereby pull the at least one strap and padded member toward the mask to secure and/or tighten the mask about the user's face.

8. The method of claim 7, wherein the at least one strap is positioned on a first side of the user's head and wherein the mask further comprises another strap that is positioned on an opposite side of the user's head.

9. The method of claim 7, wherein the tensioning device includes a rotatable knob.

10. The method of claim 7, wherein the tension member comprises lace.

11. The method of claim 7, wherein the tensioning device is positioned on the mask.

12. The method of claim 7, wherein the tensioning device is positioned on the padded member.
